(19) United States
(12) Plant Patent Application Publication
(10) Pub. No.: US 2003/0056263 P1
(43) Pub. Date: Mar. 20, 2003

(54) Chrysanthemum plant named 'Yellow Onyx Time'

(76) Inventor: Mark Roland Boeder, Den Haag (NL)

Correspondence Address: Mark K. Dickson, HOWREY SIMON ARNOLD & WHITE, LLP, 750 Bering Drive, Houston, TX, 77057-2198, US

(21) Appl. No.: 09/957,037

(22) Filed: Sep. 19, 2001

(51) Int. Cl. A01H 5/00

(52) U.S. Cl. PLT/289

(57) ABSTRACT

A new variety of Chrysanthemum plant named ‘Yellow Onyx Time,’ having an upright growth habit, a flat capitulum form and medium sized yellow decorative flowers with a green centre.

### LATIN NAME OF THE GENUS AND SPECIES

Botanical classification: Dendranthema grandiflora.

### VARIETY DENOMINATION

The new Chrysanthemum variety denomination is ‘Yellow Onyx Time.’

### BACKGROUND OF THE INVENTION

The present invention comprises a new and distinct cultivar of Chrysanthemum botanically known as Dendranthema grandiflora, and referred to by the cultivar name ‘Yellow Onyx Time’.

‘Yellow Onyx Time’, identified as 20065-1 was discovered as a natural mutation of a whole plant in a controlled cultivated planting of the variety ‘Onyx Time.’ The new variety ‘Yellow Onyx Time’ has been asexually reproduced by vegetative cuttings at Chichester, West Sussex, United Kingdom, and the distinguishing characteristics are retained through successive generations of asexual reproduction.

### BRIEF SUMMARY OF THE INVENTION

‘Yellow Onyx Time’ is a pot type of Chrysanthemum plant variety having a slightly domed capitulum form and bright yellow decorative-type flowers.

### Comparison with Parent

Plants of the new Chrysanthemum variety ‘Yellow Onyx Time’ are similar to the parent cultivar ‘Onyx Time’ in plant habitat and growth rate. In side-by-side comparisons in Chichester, West Sussex, United Kingdom, under commercial practice, plants of the new Chrysanthemum variety ‘Yellow Onyx Time’ differed from plants of the parent cultivar ‘Onyx Time’ in the following characteristics.

1. The new variety ‘Yellow Onyx Time’ produces yellow decorative type flowers whereas the parent cultivar ‘Onyx Time’ produces creamy white decorative type flowers.

2. The new variety ‘Yellow Onyx Time’ is less vigorous than ‘Onyx Time.’

3. The new variety ‘Yellow Onyx Time’ has a three day faster reaction time than ‘Onyx Time.’

4. All other characteristics of the new variety ‘Yellow Onyx Time’ appear similar to the parent variety ‘Onyx Time.’

### Comparison with Other Varieties

Plants of the new Chrysanthemum variety ‘Yellow Onyx Time’ are dissimilar to the cultivar ‘Sunny Time’ in plant habitat and growth rate. However, in side-by-side comparisons in Chichester, West Sussex, United Kingdom, under commercial practice, plants of the new Chrysanthemum variety ‘Yellow Onyx Time’ differed from plants of the cultivar ‘Sunny Time’ in the following characteristics.

1. The new variety ‘Yellow Onyx Time’ produces yellow spiky type decorative flowers, whereas the variety ‘Sunny Time’ produces a more golden yellow formal decorative flower.

2. Plants of the new variety ‘Yellow Onyx Time’ have smaller and slightly more flowers than plants of the cultivar ‘Sunny Time.’

### BRIEF DESCRIPTION OF ILLUSTRATIONS

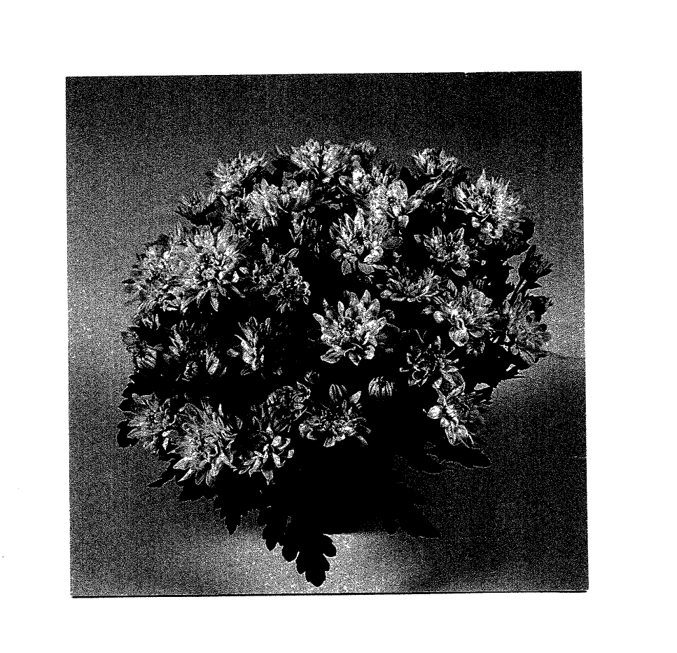

Typical specimens of the plant and flowers for the new Chrysanthemum variety ‘Yellow Onyx Time’ are shown in the accompanying photograph. The colours shown are as true as possible within the usual limits of this kind of illustration. FIG. 1 is a whole plant view of the new variety ‘Yellow Onyx Time’ grown in a pot. The plant shown in the illustration is 56 days from the start of Short Days.

### DETAILED BOTANICAL DESCRIPTION

The following description of the new Chrysanthemum variety ‘Yellow Onyx Time’ is of plants grown in a greenhouse in Chichester, West Sussex, United Kingdom in the month of June. The cultivar has not been observed under all possible environmental conditions. The phenotype may vary significantly with variations in the environment such as temperature, length of day and light intensity, without any variance in genotype. The commercial classification of the new variety is a pot Chrysanthemum.

Plants of the new variety have been grown successfully under temperature conditions averaging about 18° C. at night and about 24° C. to 25° C. during the day under light conditions of about 5,000 to 6,000 foot candles. The plants respond well to the use of growth retardant, such as three B9 treatments at about 2000 ppm. To produce a commercial product the plants may be pinched once with the centre bud removed. The typical container size for commercial growth is 14 cm. It has been observed that the shelf life of the new variety is about 22 days with a response time of about 7½weeks. The new variety is suitable for growth in a temperature range of 16° C. to 25° C.

The new variety may be produced as a spray or disbud. The following description is with respect to a plant produced as Spray pot Chrysanthemums. In the description of this new Chrysanthemum variety, colour values have been taken from The Royal Horticultural Society Colour Chart (R.H.S.C.C.).

### Plant

Plant type: Pot.

Habit: Upright and branching.

Height: Medium 18 to 20 cm.

Branching characteristics: Upright and spreading

Length of Lateral Branches: 10 to 15 cm.

Number of Breaks from Pinch: 5 to 7.

Stem Colour: 146 B.

Response Time: 52 days.

Vigour: Medium.

Shelf Life: Near 24 days.

Disease: No observations.

Pest: No observations.

Growth retardant type and treatment: B9 85% applications of 2 grams/litre at 2 days, 21 days and 31 days, after propagating unrooted vegetative cuttings.

The plants were grown for two weeks in Long Day conditions (20 hours of light) and then transferred to Short Day conditions (13 hours of dark).

Propagation:

Type.—Vegetative propagation via stem cuttings.

Time to rooting.—12-14 days with soil temperatures of 18° C.

Rooting habit.—Fine and fibrous.

### Foliage

Number of Leaves per Lateral Branch: 9 to 11.

Compound or single: Single.

Arrangement of Leaves: Alternate.

Shape of Leaf.—Typically 5 lobed, oblong ovate.

Size of Leaf.—Width (cm): Near 5½cm Length (cm): 11 cm.

Leaf Apex.—Mucronate.

Base.—Cordate.

Attachment.—Petioled.

Aspect.—Slightly undulating.

Margin.—Palmately lobed.

Surface characteristics.—Top: Pubescent Bottom: Pubescent.

Petiole: Colour: 137 B Length: 3.0 cm.

Venation: Net, prominent midvein at under side

Colour.—upper side: Near 147 D under side: Near 147 C.

Colour: Mature Leaf, upper side: Near 137 A; under side: Near 137 B Young Leaf, upper side: Near 137 B; under side: Near 137 C.

### Flower

Flower type: Decorative.

Flower form: Slightly cupped.

Flower shape: Star shaped, circular.

Number of blossoms per branch: 4 to 7.

Inflorescence form: Cyme.

Depth of fully expanded blossoms.—Near 2 cm.

Diameter of fully expanded blossoms.—Near 8½cm.

Sepals:

Number.—19 to 23.

Colour.—Near 137 A.

Length.—1.3 cm.

Width.—0.35 cm.

Texture/Appearance.—Pubescent.

Peduncles: Peduncles are more or less equal in length over all flowers and on average 5½cm.

Length.—Near 5½cm.

Colour.—Near 137 B.

Surface.—Pubescent.

Habit.—Slightly undulating.

Strength.—Medium.

Pedicel:

Length.—Terminal: Near 15½cm; Lateral: Near 15½cm.

Colour.—Near 137 B.

Surface.—Pubescent.

Habit.—Slightly undulating.

Strength.—Medium.

Ray Florets:

Form/Shape.—Straight, elliptic.

Texture/Appearance.—Matte.

Number per flower.—200 to 220.

Length.—0.5 to 3½cm; Width: 0.2 to 1.0 cm.

Apex.—Rounded and dentate.

Base.—Tapered.

Margin.—Entire.

Disc florets:

Form/Shape.—Cylindrical.

Texture/Appearance.—Shiny.

Number perflower.—10 to 14.

Length.—Near 0.2 to 0.3 cm; Width: 0.05 to 0.1 cm.

Diameter of disc.—Near 0.3 cm.

Fragrance: Faint

Flower Bud (at onset of colour):

Length.—Near 0.7 cm.

Diameter.—Near 1.0 cm.

Form/shape.—Spherical.

General flower colour: Bright golden yellow with green centre.

1. Ray florets, upper side: Immature: Near 14 A Mature: Near 14 B Older/Fading: Near 14 B.

2. Ray florets, under side: Immature: Near 14 A Mature: Near 14 B Older/Fading: Near 14 B.

3. Disc florets: Immature: Near 8 C Mature: Near 138 D Older/Fading: Near 8 C.

4. Bud: Near 138 A

Flower progression: With age flowers open slightly oblong instead of uniform circular opening. Centre of flower fades from green to yellow.

### Reproductive Organs

Ray Florets: Pistillate

Pistil number.—5.

Stigma colour.—Near 6 C.

Stigma shape.—Forked.

Style Colour.—Near 6 C.

Style Length.—Near 0.6 cm.

Disc Florets: Pistillate

Pistil number.—5.

Stigma colour.—Near 6 C.

Stigma shape.—Forked.

Style Colour.—Near 6 C.

Style Length.—Near 0.1 cm.

:Staminate.—no observations

What is claimed is:

1. A new and distinct variety of Chrysanthemum plant, substantially as described and illustrated herein.
